(19) United States
(12) Patent Application Publication
(10) Pub. No.: US 2006/0178605 A1
(43) Pub. Date: Aug. 10, 2006

(54) Tricentric orthotic hip joint and related hip brace

(76) Inventors: Herschel Sauber, Brooklyn, NY (US); James Rasmusson, Holland, MI (US); Neil B. Bergman, Plainview, NY (US)

Correspondence Address: Dara L. Onofrio, Esq.;c/o ONOFRIO LAW, Suite 1600, 1133 Broadway, New York, NY, 10010, US

(21) Appl. No.: 11/270,163

(22) Filed: Nov. 9, 2005

Related U.S. Application Data
(60) Provisional application No. 60/626,993, filed on Nov. 10, 2004.

(51) Int. Cl. A61F 5/00

(52) U.S. Cl. 602/16; 602/23; 602/24

(57) ABSTRACT

An orthotic hip joint comprising three articulating gears mounted on a support plate. The gears include a top gear; a center gear; and a bottom gear arranged in a vertical configuration wherein the gears function to permit a range of motion from 0 degrees to 90 degrees. An orthotic hip brace comprised of an upper brace for placement around the pelvic area; a lower brace for placement around the thigh area; and a hip joint comprising three articulating gears for connecting said upper and lower brace. The hip joint permits the upper brace to move with the user and does not migrate upward during sitting or bending at the hips.

This application claims the benefit of U.S. provisional application No. 60/626,993 filed Nov. 10, 2004, which is incorporated herein by reference.

### FIELD OF THE INVENTION

This invention relates to a device for supporting an injured or post-surgical hip which also provides greatly enhanced comfort during sitting or bending at the hips.

### BACKGROUND OF THE INVENTION

The human body undergoes a shortening of the chest to groin measurement when the hips and trunk are flexed. This occurs during sitting or bending over.

It has been well known to use a hip orthosis to provide support to the injured or post-surgical hip. Previous hip orthoses do not, however, allow for the shortening of the chest to groin measurement when the hips and trunk are flexed. These systems are typically monocentric, having only one axis of rotation.

A known type of hinge used in previous hip orthoses has an upper and lower member joined at an axis by a bushing screw.

In general, articulating joints for orthopedic braces, and more specifically articulating joints for knee braces are illustrated in the representative patents and publications discussed below.

U.S. Publication 2004/0024340 A1 and U.S. Pat. No. 6,589,195 both to Schwenn et al. describe orthotic braces having a modular adjustable prophylactic hip orthosis and adduction/abduction joint.

U.S. Publication 2003/0229301 A1 and U.S. Pat. No. 6,656,144 B1 both to Coligado describe articulating joints for an orthopedic brace having a movable plate rotatably connected with respect to a fixed plate.

U.S. Pat. No. 6,039,709 to Bzoch discloses an orthopedic hinge assembly incorporating two gears.

U.S. Pat. No. 5,997,493 to Young describes an orthopedic hinge with movement limitation.

U.S. Pat. No. 6,402,713 B1 to Doyle; U.S. Pat. No. 6,387,066 to Whiteside; and U.S. Pat. No. 4,632,096 to Harris disclose knee braces incorporating two gears.

U.S. Patent Publication 2004/0073149 A1 to Okediji describes an apparatus for use as an orthotic or prosthetic for assisting handicapped people to stand or walk.

U.S. Patent Publication 2002/0198481 A1 to Humphries describes an adjustment device for attachment to an orthopedic brace.

U.S. Patent Publication 2002/0157186 A1 to vanSteenburg et al. describes a hip brace apparatus.

U.S. Pat. No. 6,210,353 B1 to Barnes describes a hip brace.

U.S. Pat. No. 5,368,552 to Williamson et al. describes an orthotic hip brace.

Other known devices include U.S. Publication 2003/0100853 to Yamasaki et al. which discloses a knee brace incorporating multiple gears and guide grooves; and U.S. Pat. No. 5,421,810 to Davis et al. which describes an adjustable hip orthosis including one gear which permits adjustment of the flexion and extension of an abductor member.

As an improvement over known devices, the invention provides an orthotic hip joint and related brace that does not ride up into the user's rib cage and abdominal area when in a sitting position. More specifically, the joint includes three gears to permit extension and flexion motions with minimal movement of the brace and no discomfort by the user.

The problems associated with current hip orthosis technology include the fact that these orthoses migrate upward when the wearer bends over or sits. This is uncomfortable and potentially injurious and is particularly acute with patients who are obese. Patients who have a short trunk or a low rib cage are also prone to suffering from an orthosis that migrates upward when the hips are flexed.

The treating physician instructs the orthotist to fit the hip orthosis to provide optimum comfort for a wide range of activities. Some of the activities will be done in a standing position while some will be done in a sitting or partially bent over position.

The placement of the hip joint axis of rotation relative to the patient's anatomical axis of rotation is critical. The orthotist will typically fit the axis of the hip joint in line with the anatomical hip joint axis. Awareness of where the anatomical joint lies is accomplished by an orthotist palpating the patient to locate anatomical landmarks such as the trocanter. X-rays may also be used. Fitting a hip orthosis at the anatomical joint position allows for smooth articulation of the orthotic hip joint and minimal migration of the orthosis during ambulation. Unfortunately, with the current hip orthosis technology, joint placement at the anatomical axis will also mean that the orthosis will migrate upward when the patient bends over or sits resulting in elevation of the abdomen and impingement on the rib cage.

An orthotist may elect to lessen the affect of upward migration of the orthosis during sitting by positioning the orthotic joint distal of the anatomical joint. This will enhance the comfort of the patient during sitting, but it will also compromise the smooth action of the joint during ambulation, causing the orthosis to move relative to the skin with each step taken. This in turn causes rubbing of the skin which leads to abrasions and ulcerations.

The invention disclosed herein is a hinge for a hip orthosis that has three axes of rotation. The three axes relate to each other in an exacting manner due to their being connected by means of gears. There is a large gear at the bottom, a small gear at the top and an idler gear in-between.

When the hinge is flexed, the idler gear ‘walks’ down the large gear, bringing with it the small gear which in turn pulls the pelvic section of the orthosis down via the extension arm bar that is integral with the small gear. This shortening continues over 90 or more degrees of flexion.

### SUMMARY OF THE INVENTION

The present invention overcomes the problems of prior devices by providing a hip brace with a tricentric hinge that allows the pelvic section of the orthosis to not migrate upward during sitting or bending at the hips.

In particular, the orthotic hip joint comprises three articulating gears, namely a top gear, a center gear and a bottom gear mounted on a support plate and arranged in a vertical configuration.

The top gear is comprised of a semi-circular bottom portion with triangular grooves around its outer edge and a top portion which extends to form a pelvic extension arm.

The center gear is circular and has triangular grooves around its entire circumference.

The bottom gear comprises an enclosed U-shaped body. The top portion of the U-shaped body comprises triangular grooves and the bottom portion extends to form a thigh extension arm.

The top and center gears each have a small holed circle within the middle of the gears. The bottom gear has a small holed circle toward its lower end.

All three gears function to permit a range of motion from 0 to 90 degrees.

In another embodiment of the invention, an orthotic hip brace is comprised of an upper brace for placement around the pelvic area, a lower brace for placement around the thigh area and a hip joint comprising the three articulating gears that connect the upper and lower brace.

The hip joint in the brace includes a support plate which may contain adjustment holes along its periphery for extension and flexion stops. The top, center and bottom gears are mounted on the support plate. The hip joint also includes a pelvic extension arm for attachment to the upper brace and a thigh extension arm for attachment to the lower brace.

The upper brace of the orthotic hip brace moves with the user and does not migrate upward during the sitting or bending at the hips.

The hip joint is preferably made of stainless steel or aluminum.

Other objects, features and advantages of the present invention will be apparent when the detailed description of the preferred embodiments of the invention are considered with reference to the drawings, which should be construed in an illustrative and not limiting sense as follows:

### BRIEF DESCRIPTION OF THE DRAWINGS

FIG. 1 is an illustration of the hip orthosis of the invention in which the user is in a standing position with the invention hip joint in full extension;

FIG. 2 is an illustration of the hip orthosis of the invention in which the user is in a sitting position with the invention hip joint at 90°;

FIG. 3 is an illustration of the hip joint of the invention in full extension;

FIG. 4 is an illustration of the hip joint of the invention at 30°;

FIG. 5 is an illustration of the hip joint of the invention at 60°;

FIG. 6 is an illustration of the hip joint of the invention at 90°;

FIG. 7 is an illustration of the hip joint of the invention at 10°; and

FIG. 8 is a schematic illustration detailing the gears of the hip joint according to the invention.

### DETAILED DESCRIPTION OF THE INVENTION

In accordance with the present invention, a hip joint is provided for a hip orthosis that offers the necessary support to the injured or post-surgical hip and that also eliminates the problem of the pelvic band migrating upward when the patient sits or bends at the waist. The migration of the pelvic band is uncomfortable, especially for obese patients, displacing the abdomen upward and impinging on the patient's rib cage.

The hip joint of the invention affords comfort for the wearer of a hip orthosis during sitting by allowing for the fact that the chest to groin measurement decreases when the hips are flexed. The hip joint systems of the invention are designed to provide from 1.0″ to 2.0″ of shortening over 90 degrees of flexion.

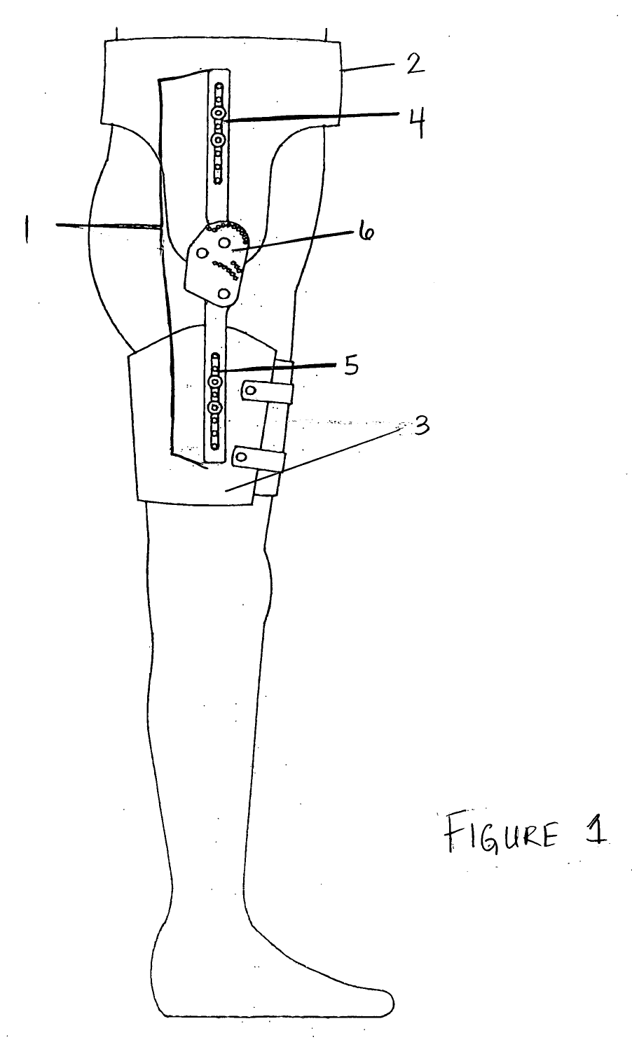

FIGS. 1-7 illustrate how the gears of the hip joint according to the invention move and are also representative of various positions.

In general, FIG. 1 represents the hip orthosis of the invention in which the user is in a standing position with the hip joint in full extension. In particular the hip orthosis is comprised of a hip joint 1, an upper brace 2 and a lower brace 3. The hip orthosis contains a pelvic extension arm 4 for attachment of the hip joint 1 to the upper brace and a thigh extension arm 5 for attachment to the lower brace 3.

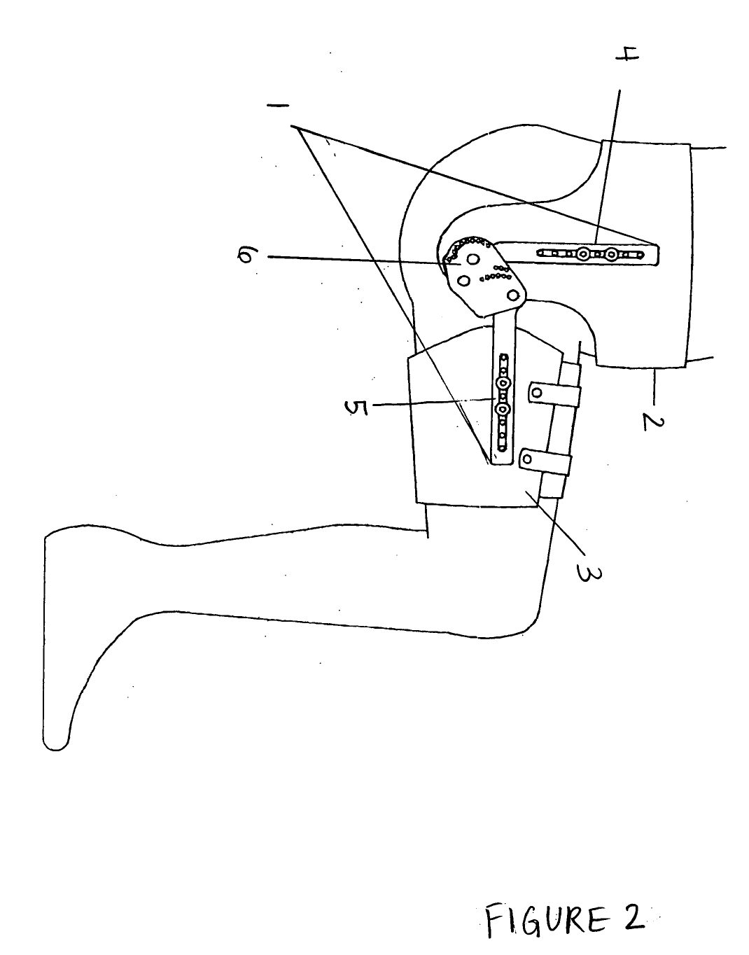

FIG. 2 represents the hip orthosis of the invention in which the user is in the sitting position with the hip joint at 90°

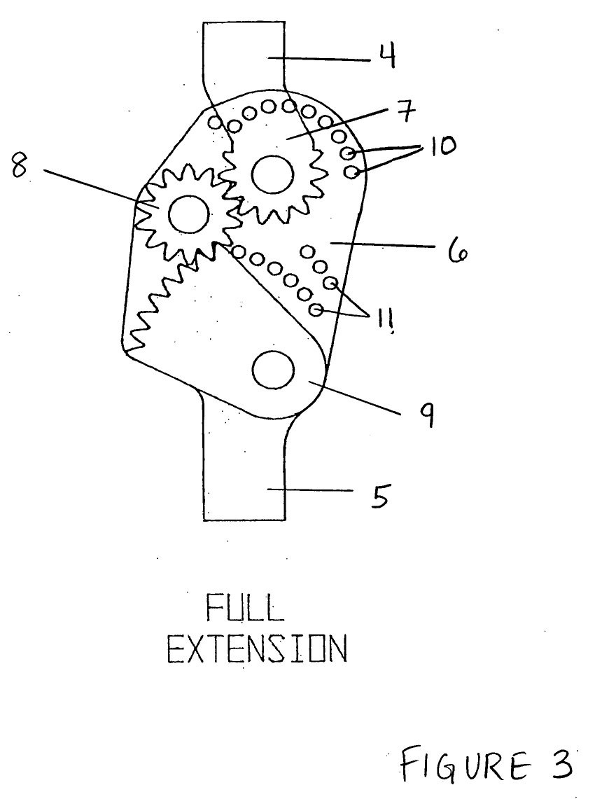

FIG. 3 illustrates the hip joint 1 of the invention in full extension. The hip joint 1 comprises the support plate 6 upon which three articulating gears, namely, the top gear 7, center gear 8 and the bottom gear 9 are mounted and arranged in a vertical configuration. The support plate 6 may contain adjustment holes at various locations along its periphery for predefined extension stops 10 and flexion stops 11 of the joint.

The top gear 7 is comprised of a semi-circular bottom portion with triangular grooves around its outer edge which engage the center gear 8. The top gear 7 may also contain a hole in its center. The pelvic extension arm 4 extends from the top portion of the top gear for attachment of the upper brace.

The center gear 8 is circular with triangular grooves around its entire circumference. The center gear 8 may also contain a hole in its center.

The bottom gear 9 comprises an enclosed U-shaped body. The top portion of the U-shaped body comprises triangular grooves which engage the center gear 8 and the bottom portion extends to form the thigh extension arm 5. The bottom gear 9 may also contain a small hole towards its lower end.

The top gear 7, the center gear 8 and the bottom gear 9 function to permit a range of motion from 0 to 90 degrees. Both the top gear 7 and the bottom gear 9 are drive gears and have extension arms that are integral with the gear. The extension arms function as attachment bars for connecting the joint to body-form components, typically a thigh section and a pelvic section.

The two drive gears have different diameters, with a 3:1 ratio being typical. The top gear 7 has a diameter generally smaller than bottom gear 9.

The center gear 8 is an idler gear which serves the purpose of reversing the direction of rotation as well as being the component that directs the shortening of the joint. When the hip joint 1 is flexed, the center gear 8 ‘walks’ down the bottom gear 9, bringing with it the top gear 7 which in turn pulls the pelvic section of the orthosis down via the pelvic extension arm 4. This shortening continues over 90 or more degrees of flexion.

The hip joint 1 of the invention is preferably made of materials such as stainless steel or aluminum. However, other materials may be used that are sturdy, lightweight and able to permit the range of motion encompassed by the invention.

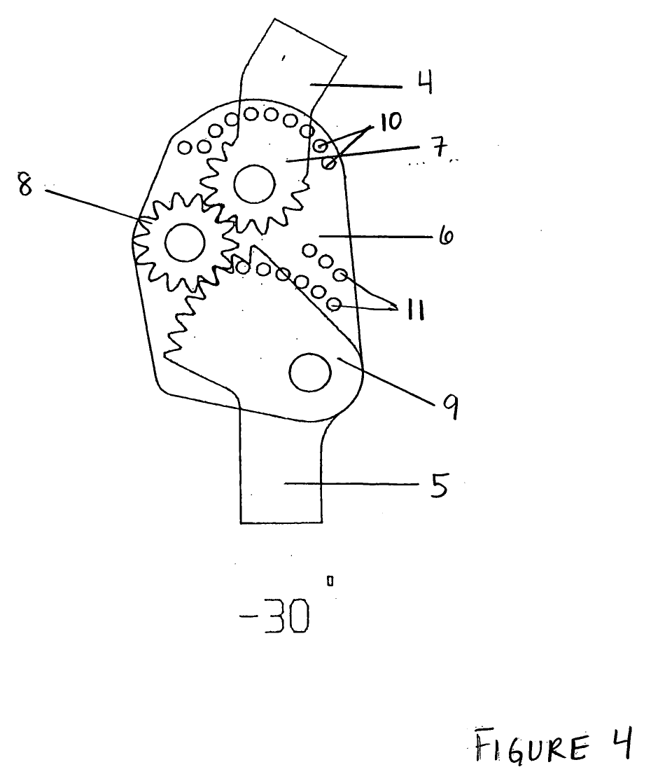

FIG. 4 represents the hip joint at a flexion of 30° wherein the top gear 7 and bottom gear 9 have rotated around the center gear 8.

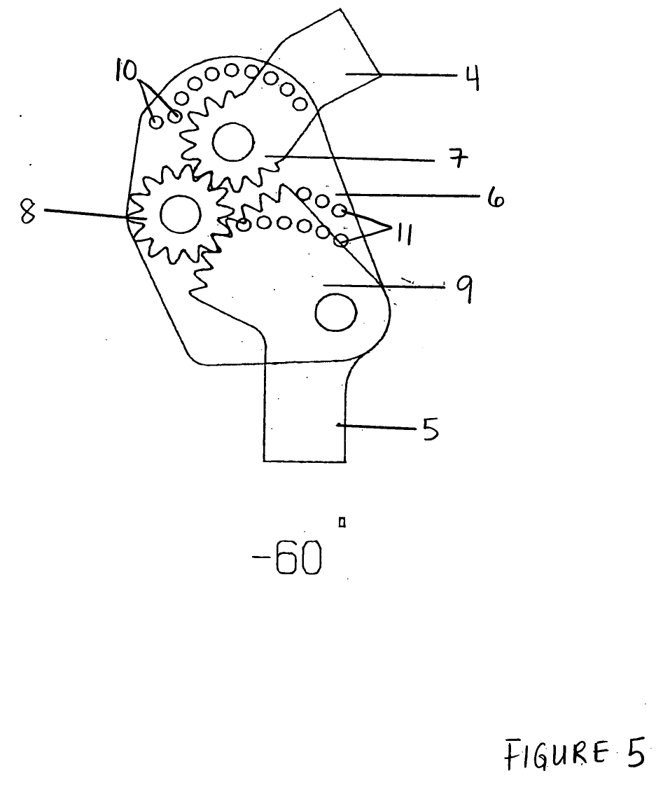

FIG. 5 represents the hip joint at a flexion of 60° wherein the top gear 7 and bottom gear 9 have rotated around the center gear 8.

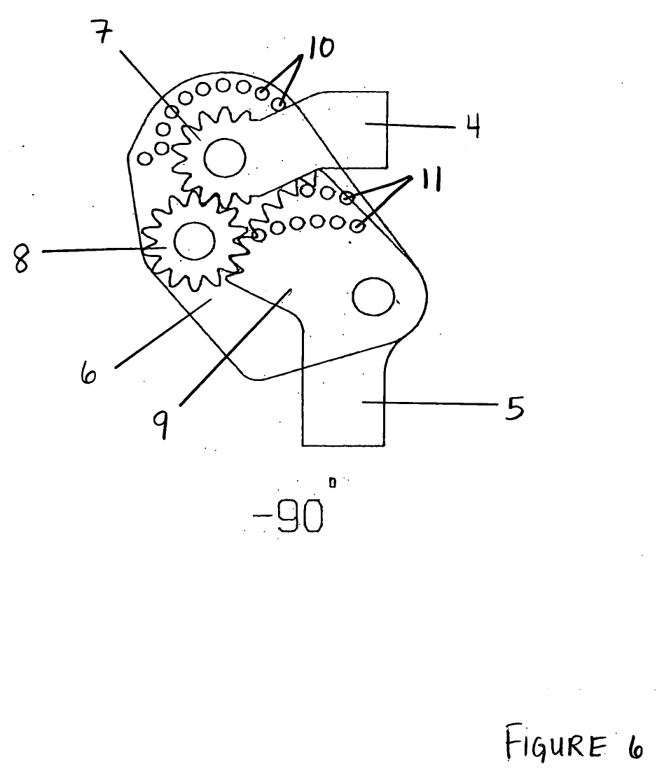

FIG. 6 represents the hip joint at a flexion of 90° wherein the top gear 7 and bottom gear 9 have rotated around the center gear 8.

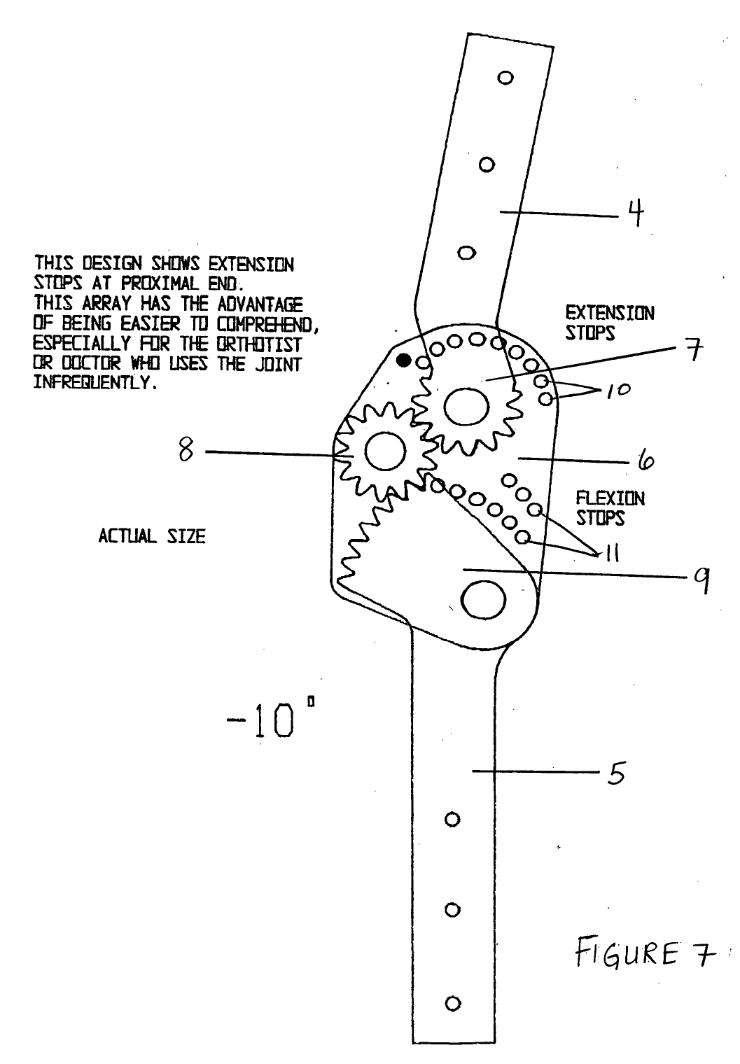

FIG. 7 represents the hip joint at a flexion of 10° wherein the top gear 7 and bottom gear 9 have rotated around the center gear 8. In this figure the extension stops at the proximal end.

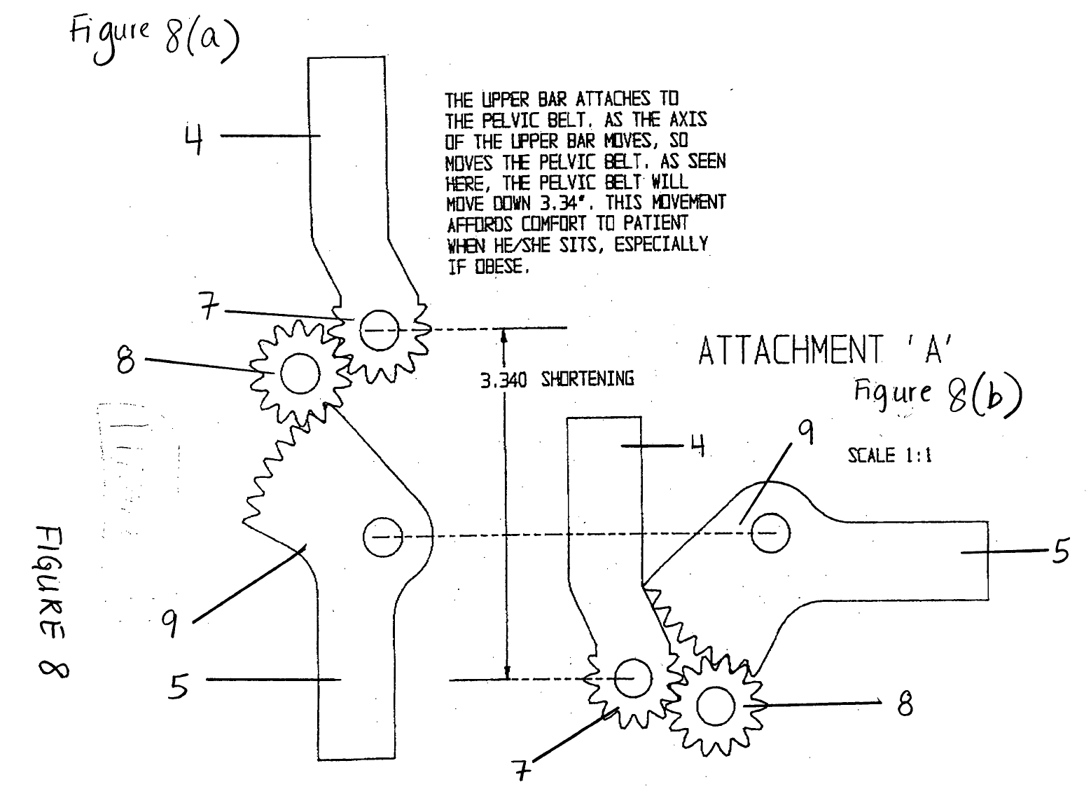

FIG. 8 illustrates a schematic illustration of the hip joint detailing the movement of the gears. FIG. 8(a) illustrates the hip joint in full extension. FIG. 8(b) represents hip joint when a person is sitting at 90°. The pelvic extension arm 4 attaches to an upper pelvic belt 2. As the axis of the pelvic extension arm 4 moves, the upper brace 2 will move down approximately 3.34″ thereby providing comfort to the patient when he or she is sitting.

The advantage of the hip joint is that it affords comfort for the wearer of a hip orthosis during sitting by allowing for the fact that the chest to groin measurement decreases when the hips are flexed. The hip joint system can be designed to provide from 1.0″ to 2.0″ of shortening over 90 degrees of flexion.

The foregoing description of various and preferred embodiments of the present invention has been provided for purposes of illustration only. The invention now being fully described, it will be apparent to one of ordinary skill in the art that many changes and modifications can be made thereto without departing from the spirit and scope of the invention as set forth herein and in the following claims.

What is claimed is:

1. An orthotic hip joint comprising three articulating gears mounted on a support plate

2. The orthotic hip joint according to claim 1, wherein said gears comprise a top gear; a center gear; and a bottom gear arranged in a vertical configuration.

3. The orthotic hip joint according to claim 1, wherein said top gear is comprised of a semi-circular bottom portion with triangular grooves around the outer edge of said semi-circular portion; and a top portion which extends to form a pelvic extension portion.

4. The orthotic hip joint according to claim 1, wherein said center gear is circular with triangular grooves around the entire circumference of said gear.

5. The orthotic hip joint according to claim 1, wherein said bottom gear comprises an enclosed U-shaped body;

6. The orthotic hip joint according to claim 5, wherein the top portion of said U-shaped body comprises triangular grooves; and a bottom portion which extends to form a thigh extension portion.

7. The orthotic hip joint according to claim 1, wherein said top and center gears further comprise a small holed circle within the middle of said gears.

8. The orthotic hip joint according to claim 1, wherein said bottom gear further comprises a small holed circle towards the lower end of said gear.

9. The orthotic hip joint according to claim 1 wherein said gears function to permit a range of motion from 0 degrees to 90 degrees.

10. An orthotic hip brace comprised of an upper brace for placement around the pelvic area; a lower brace for placement around the thigh area; and a hip joint comprising three articulating gears for connecting said upper and lower brace.

11. The orthotic hip brace according to claim 10 wherein said hip joint includes a support plate which contains adjustment holes along the periphery of said support plate for extension and flexion stops.

12. The orthotic hip brace according to claim 11 wherein said gears comprise a top, center and bottom gears mounted on said support plate.

13. The orthotic hip brace according to claim 10 wherein said upper brace of claim 10 moves with the user and does not migrate upward during sitting or bending at the hips.

14. The orthotic hip brace according to claim 10, wherein said hip joint further comprises a pelvic extension arm for attachment to the upper brace.

15. The orthotic hip brace according to claim 10, wherein said hip joint further comprises a thigh extension arm for attachment of said lower brace.

16. The orthotic hip brace according to claim 10, wherein said hip joint is comprised of stainless steel or aluminum.
